(19) United States
(12) Patent Application Publication
(10) Pub. No.: US 2005/0026867 A1
(43) Pub. Date: Feb. 3, 2005

(54) Hyaluronic acid-associated superoxide dismutase mimics and their use in the treatment of joint pain

(75) Inventor: James J. Benedict, Arvada, CO (US)

Correspondence Address: WILLIAMS, MORGAN & AMERSON, P.C./ZIMMER, 10333 RICHMOND, SUITE 1100, HOUSTON, TX, 77042, US

(73) Assignee: Zimmer Orthobiologics, Inc.

(21) Appl. No.: 10/916,108

(22) Filed: Aug. 11, 2004

Related U.S. Application Data
(63) Continuation of application No. 10/172,624, filed on Jun. 14, 2002.

(51) Int. Cl. A61K 31/728; A61K 31/555

(52) U.S. Cl. 514/54; 514/185

(57) ABSTRACT

Herein is disclosed a composition, comprising a superoxide dismutase mimic (SODm) associated with hyaluronic acid (HA), wherein the composition is a liquid at about 37° C. The composition is useful in a method of treating pain or inflammation in a joint of a mammal, comprising (i) providing the composition and (ii) injecting the composition into or close to the joint.

### BACKGROUND OF THE INVENTION

1. Field of the Invention

The present invention relates generally to the field of treatment of pain and inflammation in mammalian joints. More particularly, it concerns compositions comprising a biomolecule and an associated free radical scavenger, and the use of such compositions in treating pain or inflammation. Still more particularly, it concerns compositions comprising a scaffold molecule, such as hyaluronic acid, collagen, and collagen derivatives such as gelatin, polyethylene oxide, polyethylene glycol, or polypropylene glycol, wherein the scaffold molecule is associated with superoxide dismutase mimics, and the use of such compositions in the treatment of pain and inflammation in mammalian joints.

2. Description of Related Art

A number of human and animal diseases have as symptoms pain and inflammation of the joints. One such disease is rheumatoid arthritis, a common human autoimmune disease characterized by chronic inflammation of the synovial joints (such as the knee) and by subsequent progressive destruction of articular tissue. A second such disease is osteoarthritis, which has similar symptoms. One component of the synovial joints that is degraded in both rheumatoid arthritis and osteoarthritis is hyaluronic acid (HA).

HA is a linear polysaccharide comprising repeating glucuronic acid and N-acetyl glucosamine disaccharide units. HA has a high molecular weight (up to and exceeding 5-7×10⁶ Da, although HA species having molecular weights from about 1 ×10⁴ Da and higher can be used in compositions and methods of the present invention) and readily associates with water. Though not to be bound by any particular theory, these properties of HA are believed to impart HA solutions with high viscoelasticity, even at low concentrations of HA.

HA constitutes a significant component of a number of bodily tissues and fluids, such as the extracellular matrix of cartilage, synovial fluid, and the vitreous humor of the eye. The terms “hyaluronic acid” and “HA” as used herein are interchangeable. Moreover, the terms are intended to encompass different molecular weights of the compound, depending upon the number of repeating monomer units, and the terms also include different forms of the molecule, such as the acid form and the salt form, as well as the neutral form. HA is also referred to in the literature as “hyaluronan.”

As stated above, in osteoarthritis and rheumatoid arthritis, the concentration and molecular weight of HA in the joint fluid are reduced by mechanisms attributed, at least in part, to attack on HA by reactive oxygen species such as superoxide (O₂⁻) found in inflamed joints.

Based on these observations, several attempts have been made to treat rheumatoid arthritis, osteoarthritis, and similar diseases by administering hyaluronic acid to supplement the degraded or damaged HA in the arthritic joint. HA has been administered to patients in Europe since 1987. In the United States, three approved commercially available products are known (Synvisc by Biomatrix/Genzyme, Hyalgan by Fidia, and Supartz by Seikagaku). HA is usually injected into the knee or other joint to act as a viscosupplement. However, these treatments have a disadvantage in that the half-life of hyaluronic acid in the joint (which typically contains superoxides that can cleave the HA molecule), based on rabbit studies with labeled HA, is in the range of about 10-13 hr (Brown et al., Exp. Physiol. 76:125-134 (1991)); therefore, repeat or supplemental injections of HA are required to replace injected HA that is degraded or cleared by physiological processes or reactive oxygen species. It is desirable to have injectable formulations of HA that are retained for longer times than the HA formulations described above.

Although HA is a particularly preferred scaffold molecule in embodiments of the present invention, other biomolecules and synthetic scaffold molecules known in the art can be used. Included among the former group are collagen and collagen derivatives such as gelatin, and included in the latter group are polyethylene oxides (PEO), polyethylene glycols (PEG) and polypropylene glycols (PPG), which are biocompatible and can be formulated as a liquid suitable for local administration via, for example, a syringe.

In addition to degrading HA, superoxides are believed to play a role in perpetuating the chronic inflammatory state associated with rheumatoid arthritis. This hypothesis has been supported by studies in both animal models of arthritis and pilot experiments carried out in patients with active rheumatoid arthritis. Based on these observations, there have been several attempts to treat rheumatoid arthritis, osteoarthritis, and similar diseases by administering superoxide dismutase (SOD), an enzyme which catalyzes the dismutation of superoxide.

One such product, Orgotein®, a bovine SOD with a Cu/Zn catalytic site, was used in preliminary clinical trials in patients with inflammatory disorders including rheumatoid arthritis and osteoarthritis. In patients with active classical rheumatoid arthritis, intraarticular injection of Orgotein® ameliorated a number of symptoms, as evidenced by improved rheumatoid arthritis activity index (morning stiffness, flexion range, pain, walking time), decrease in the level of rheumatoid factor, reduced intake of rescue acetaminophen, and overall improvement in physician and patient global ratings. Similar benefits were seen in patients with osteoarthritis (McIlwain et al., Am. J. Med. 87:295-300 (1989)). However, Orgotein® was removed from the market because of the development of immune responses against the bovine enzyme. Use of SOD is also complicated by solution instability, bell-shaped dose response curves, high susceptibility to proteolytic digestion, and limited cell and organ penetration.

An alternative approach involves the use of superoxide dismutase mimics (SODms) in place of SOD itself. A SODm is a low molecular weight compound that catalyzes the dismutation of superoxide at a rate comparable to that of the native enzyme. Udipi et al., “Modification of inflammatory response to implanted biomedical materials in vivo by surface bound superoxide dismutase mimics,” J. Biomed. Mater. Res., pp. 550-560 (2000) reports the use of SODms consisting of a Mn(II) complex of a macrocyclic polyamine ring, wherein the SODms were covalently linked to small disks of ultra-high molecular weight polyethylene, poly(etherurethane urea), or tantalum metal and implanted in a subcutaneous rat implant model. Anti-inflammatory effects were seen for the SODm-linked implants relative to control implants without SODm. Salvemini et al., Science 286:304-306 (1999), reports the use of a Mn(II) complex with a bis(cyclohexylpyridine)-substituted macrocyclic ligand in inhibiting edema in a rat inflammation model and in increasing survival time in a rat splanchnic artery occlusion model.

### SUMMARY OF THE INVENTION

In one embodiment, the present invention relates to a composition, comprising a superoxide dismutase mimic (SODm) associated with hyaluronic acid (HA), wherein the composition is a liquid at about 37° C.

In another embodiment, the present invention relates to a method of treating pain or inflammation in a joint of a mammal, comprising (i) providing a composition, comprising a superoxide dismutase mimic (SODm) associated with hyaluronic acid (HA), wherein the composition is a liquid at about 37° C. and (ii) delivering the composition into or close to the joint. In a preferred embodiment, the composition is delivered by injection in an amount sufficient to ameliorate at least one symptom of an inflamed joint selected from the group consisting of: superoxide levels in the joint, rheumatoid arthritic factor index, flexion range, morning stiffness, and pain levels.

In another embodiment, the prevent invention relates to a method of preventing or suppressing ostephyte formation in an inflamed joint of a mammal. The method comprises (i) providing a liquid composition comprising a SODm associated with hyaluronic acid and (ii) delivering the composition to a joint of a mammal.

The present invention preferably allows relief of pain and inflammation in a joint for a relatively extended time, with minimal potential for immune responses and other undesirable side effects. The present invention provides injectable formulations of HA that are retained for longer times as an effective viscosupplement than previously known HA formulations. The present invention also provides compositions comprising a SODm that are useful in treating arthritis.

### DESCRIPTION OF ILLUSTRATIVE EMBODIMENTS

In one embodiment, the present invention relates to a composition comprising a superoxide dismutase mimic (SODm) associated with hyaluronic acid (HA), wherein the composition is a liquid. More particularly, the composition preferably comprises a liquid at about body temperature (37° C.).

As noted, HA is a linear polysaccharide comprising repeating glucuronic acid and N-acetyl glucosamine disaccharide units. It is a significant component of a number of bodily tissues and fluids, e.g., the extracellular matrix of cartilage, synovial fluid, and the vitreous humor of the eye. HA can be isolated from mammalian and avian tissues or certain strains of cultured bacteria, as is known in the art. HA (as a sodium salt isolated from bacteria) is commercially available from Lifecore (Chaska, Minn.), among other suppliers.

Superoxide dismutase mimics (also referred to in the literature as SOD synzymes or SOD mimetics) are low molecular weight, nonpeptidyl compounds comprising either Mn(II) or Cu/Zn and having the property of catalyzing the conversion of superoxide to hydrogen peroxide, according to the following reaction (Equation 1):
2O₂⁻+2H⁺→O₂+H₂O₂ (Eq. 1)

By “low molecular weight” is meant a molecular weight of less than about 2000 Da. In one embodiment, the molecular weight is from about 400 Da to about 600 Da. The low molecular weights associated with SODms is in sharp distinction to wild-type or mutated superoxide dismutase enzymes known in the art, which typically have molecular weights from about 32 kDa to about 135 kDa.

In general, the SODm comprises a macrocyclic compound having multiple amine functional groups and an active catalytic center, which may comprise Mn, Cu/Zn, or other catalytic groups capable of catalyzing superoxide to hydrogen peroxide according to Equation 1. More particularly, the SODm may comprise a tetra-amine, pentamine, or hexamine Mn complex macrocycle, with the macrocycle complex containing from 10-30 atoms, exclusive of additional functional groups.

In one embodiment, the SODm comprises a pentamine macrocycle and Mn(II). In a preferred embodiment, the macrocycle comprises from 14-16 atoms, excluding additional substituents which may be bound thereto. By “pentamine macrocycle” is meant a structure comprising two or more cyclic or fused cyclic moieties and a total of five nitrogens in the form of either —NH—, —N═, or any combination thereof, wherein all five nitrogens are capable of coordinating with Mn(II). By way of example, one approach to the production of the SODm is computer aided design, as discussed by Aston et al., Inorg. Chem. 40:1779-1789 (2001). In one embodiment, the SODm comprising a pentamine macrocycle and Mn(II) has structure I:

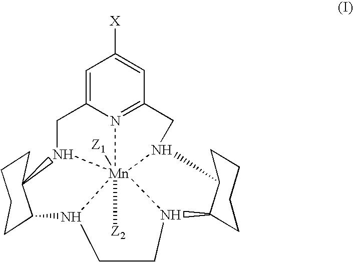

wherein X is selected from the group consisting moieties comprising —H, moieties comprising —NH₂, moieties comprising —COOH, and moieties comprising —CHOH; and Z₁ and Z₂ are independently -halide. In one embodiment, X is —S—(CH₂)₂—NH₂, and Z₁ and Z₂ are —Cl.

More generally, structure I may be modified in various ways without destroying the ability of the modified compound to catalyze the dismutation of superoxide to hydrogen peroxide according to Equation 1. All such structures may be coupled to liquid HA for administration to an inflamed joint, and are considered to be within the scope of the present invention so long as catalytic activity for dismutation of superoxide is retained. In particular, the X substituent on the pyridine ring may be in any of positions 3, 4, or 5, rather than position 4 of the ring as depicted in FIG. 1. Similarly, the two cyclohexyl groups coupled to the macrocycle may comprise other substituents. One such group could be an ethylene group, resulting in an ethylenediamine group. Other groups are possible, and are considered within the scope of the invention so long as the foregoing catalytic activity is preserved. Further, the two carbon/two nitrogen substituent shown directly across the structure from the pyridine ring (i.e., adjacent component Z₂) could have substituents on either or both of the carbon atoms. Additionally, the steric location of the two amine groups on the cyclohexyl rings allows for stereoisomers to exist. These stereoisomers, along with the other modifications to structure I discussed above, may have different levels of catalytic activity for superoxide dismutation. However, so long as some level of catalytic activity is retained, the compositions are considered to be within the scope of the invention. The foregoing modifications are depicted in structure II:

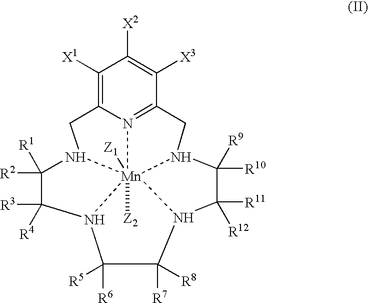

As will be appreciated by persons of skill in the art, other SODm structures could be coupled with HA or another scaffold molecule to yield a liquid composition suitable for local administration in the body of a patient. Such structures, and additional details concerning the structure, function, and activity of SOD and SODm are provided in U.S. Pat. Nos. 5,227,405, 5,637,578, 5,994,339, 6,084,093, 6,204,259, and 6,214,817, each of which is hereby incorporated by reference herein in its entirety.

In addition to HA and SODm, compositions according to the present invention can comprise additional components. Typically, the composition comprises a solvent selected from water or an aqueous buffer such as Tris buffer, especially 5 mM Tris, or HEPES buffer, especially 60 mM HEPES. The pH of the aqueous buffer is preferably from about 5 to about 9, more preferably from about 6 to about 8, and even more preferably from about 7 to about 7.5. Strongly acidic solvents can promote dissociation of the metal ion (e.g., Mn) from the SODm ring.

The composition can further comprise adjuvants, preservatives, or other compounds which aid the association of HA and SODm, storage stability of the composition, or the intended use of the composition. An exemplary use of the composition is described below.

The concentration of HA in water or aqueous buffer is preferably in the range of about 0.01 w/v % HA to about 20 w/v % HA, preferably about 0.1 w/v % HA to about 10 w/v % HA. A concentration of 1 w/v % HA has been found to be useful. The concentration of SODm in the water or aqueous buffer is preferably in the range of about 1 mg/mL to about 200 mg/mL, preferably from about 5 mg/mL to about 100 mg/mL, more preferably from about 5 mg/mL to about 30 mg/mL, more preferably still from about 10 mg/mL to about 20 mg/mL. As stated, the composition is a liquid at about 37° C. Because the composition is a liquid at this temperature, it is typically liquid at room temperature (about 20-22° C.) or refrigerator temperature (about 4° C.), and thus is conveniently processed, stored, and administered. As a liquid at about 37° C., such as from about 30° C. to about 45° C., the composition is liquid at the body temperature of humans or other mammals and thus would remain liquid, as opposed to a solid, when injected into a human or other mammal as can be done in accordance with the method described below.

As stated, the HA and SODm of the composition are associated. By “associated” is meant that a molecule of HA is in close proximity to (i.e., less than 100 nm from) a molecule of SODm. This association can be a result of physical proximity (such as by dissolution of both HA and SODm in a solution), by van der Waals attractive forces, or by chemical bonding, among other forces. The association may comprise a photo-initiated coupling to the HA of an appropriate SODm agent.

In one embodiment, the association between HA and SODm is as a result of a chemical bond between HA and SODm. The chemical bond can be ionic or covalent (including coordinate covalent). An ionic bond, as that term is used herein, is one in which a first charge on an HA molecule is attracted to a second, opposite charge on a SODm molecule. A covalent bond, as that term is used herein, is one in which at least one atom which is a constituent of the HA molecule shares electrons in a bonding orbital with at least one atom which is a constituent of the SODm molecule.

In one embodiment, the chemical bond between HA and SODm is a covalent bond. In a further embodiment, the covalent bond is selected from the group consisting of an amide bond, an ester bond, and an ether bond. By “amide bond” is meant a —CONH— moiety, wherein at least one atom, and typically the —C(═O)— moiety or the —NH— moiety, is derived from the HA and at least one atom, and typically the other of the —C(═O)— moiety or the —NH— moiety than is derived from the HA, is derived from the SODm. By “ester bond” is meant a —COO- moiety, wherein at least one atom, and typically the —C(═O)— moiety or the —O— moiety, is derived from the HA and at least one atom, and typically the other of the —C(═O)— moiety or the —O— moiety than is derived from the HA, is derived from the SODm. By “ether bond” is meant an —O— moiety, wherein the oxygen atom is derived from either the HA or the SODm and the oxygen atom is bound to both an atom of the HA and an atom of the SODm. In most embodiment of the invention, a covalent bond is preferred.

In another embodiment, the present invention relates to a method of treating pain or inflammation in a joint of a mammal, comprising:
(i) providing a composition, comprising a superoxide dismutase mimic (SODm) associated with hyaluronic acid (HA), wherein the composition is a liquid at about 37° C.; and,
(ii) injecting the composition into or close to the joint.

Any pain having its origins in physiological events in the joint, and any inflammation of the joint or tissues comprising or in proximity to the joint, is treatable by the method. Typical symptoms of inflammation include, but are not limited to, redness, swelling, soreness, weakness, fluid accumulation, and partial loss of motion, among others. Not all of these typical symptoms need be present for one of ordinary skill in the art to recognize that inflammation has occurred. Use of the word “or” in the phrase “pain or inflammation” is in the inclusive sense, i.e., the phrase “pain or inflammation” as used herein is equivalent to the phrase “pain, inflammation, or both.” By “treating” is meant a reduction in the intensity of pain or the extent of inflammation to levels below those experienced prior to the performance of the method. “Treating” may also comprise the prevention of an increase in pain or inflammation. It will be understood that some subjects of the method may not experience a reduction in pain intensity or inflammation extent, but if one of ordinary skill in the art anticipated a reasonable likelihood of success in treating pain or inflammation in these subjects by performing the method, then that performance of the method is within the scope of the present invention.

This method is applicable to the treatment of any mammal, and is especially applicable to the treatment of a human. Exemplary conditions which can be ameliorated by use of this method include, but are not limited to, osteoarthritis and rheumatoid arthritis, among others.

Any joint found in the musculoskeletal structure of the mammal can be the target of the injection, as will be described below. Exemplary joints include, but are not limited to, articulating joints in the hip, the knee, the ankle, the shoulder, the elbow, the wrist, the neck, finger joints, toe joints, and vertebral joints, among others. Any other joint at which pain or inflammation occurs can be the target of the injection as well. In one embodiment, the joint is the knee. In another embodiment, the joint is a facet joint between vertebral bodies of the spine.

The method comprises providing a composition comprising a superoxide dismutase mimic (SODm) associated with hyaluronic acid (HA), wherein the composition is a liquid at about 37° C. This composition has been described above. In one embodiment, the composition is sterilized by an appropriate technique, such as heating, filtration, or exposure to ultraviolet light, and combinations of the foregoing, among others. Aseptic formulation techniques are also possible.

The injecting step involves injecting the composition into or close to the joint. By “into” the joint is meant injection into an area defined by bone, cartilage, ligament, or tendon and regarded as the joint by one of ordinary skill in the art. In a particular embodiment, injection comprises injecting the composition into the joint capsule. A joint typically contains a fluid, e.g., the synovial fluid of the knee, and thus injection into the joint is typically injection into the fluid. By “close to” the joint is meant a subcutaneous or intramuscular injection within an area of redness or swelling within, typically, 5 cm or less of the joint. Preferably, the composition is injected directed into the joint capsule, which maintains the composition within the capsule and minimizes and/or retards systemic migration or delivery to other parts of the body. Localized delivery may be achieved by injecting the composition into the joint capsule. Injecting involves techniques known in the art.

The dose of the injected composition is preferably from about 0.0001 mg SODm/kg body weight to about 10 mg SODm/kg body weight. In one embodiment, the dose is from about 0.1 mg SODm/kg body weight to about 5 mg SODm/kg body weight.

By performing the method, a composition comprising HA and SODm is introduced into the joint. HA is a component of the fluid contents of several joints, such as the synovial fluid of the knee joint, and in a number of conditions, such as osteoarthritis and rheumatoid arthritis, HA naturally present in the joint degrades. As discussed above, prior art techniques of injecting HA in the absence of SODm have been unsuccessful, presumably because the injected HA also degrades. Though not to be bound by theory, it is believed that degradation of HA occurs as a result of free radical attack, e.g. attack by superoxide, with the free radicals generated by physiological processes associated with inflammation of the joint. The present method of injecting a composition comprising SODm in addition to HA provides a component (viz., SODm) that counteracts the generation of free radicals by catalyzing the dismutation of superoxide as shown by Eq. 1, above. As a result, the injected HA is expected to undergo degradation more slowly. Further, the action of SODm in dismutation of superoxide is expected to have other beneficial effects by ameliorating other negative effects of superoxide and other free radicals in the inflammation response. In addition, SODm is not expected to give rise to immunogenic responses, as have been reported from the use of bovine superoxide dismutase.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

A preclinical study of HA and HA-SODm was performed.

Materials and Methods

Hyaluronic acid (HA) used was medical grade with a nominal molecular weight of 1.2 million Da and an intrinsic viscosity of 20 dl/g (Lifecore, Chaska, Minn.). The SODms used had structure I, as described above. A first SODm, designated 403, had structure I, wherein X was —H and Z₁ and Z₂ were both —Cl. A second SODm, designated 470, had structure I, wherein X was —S—(CH₂)₂—NH₂ and Z₁ and Z₂ were both —Cl. The SODms were synthesized according to the procedure given by Udipi et al., J. Biomed. Mater. Res. 51:549-560 (2000).

HA was covalently bound to SODm 470, to produce HA-SODm, following the procedure of Bulpitt et al., J. Biomed. Mater. Res. 47:152-169 (1999). To a solution of 1.003 g (2.5 mmol) sodium salt of hyaluronic acid in 220 mL distilled water was added 1.4 g (2.5 mmol) of SODm 470. The pH of the solution was lowered from 9.3 to 6.8 by careful addition of 0.1 M HCl. A solution of 1-(3-dimethylaminopropyl)-3-ethylcarbodiimide hydrochloride (EDC.HCl) (0.24 g, 1.25 mmol) and 1-hydroxy-7-azabenzotriazole (HOAT), (0.17 g, 1.25 mmol) in dimethylsulfoxide (DMSO)/water (2.0 mL, 1:1 v/v) was added and the pH was adjusted from 4.5 to 6.8 and maintained at 6.8 by incremental addition of 0.1M NaOH. The contents were stirred overnight at ambient temperature. After 20 hr, the pH was readjusted to 6.8 from 6.94, and again stirred for a total of 48 hr. Then, the pH was adjusted to 7.0 and dialyzed in Pierce Slide-a-dialyzer cassettes (MW cutoff, 10,000) against distilled water for 40 hr. Dialyzed cassette contents were extracted by syringe (about 300 mL vol) and 15 g NaCl was added to obtain a 5% salt solution. The reaction product was precipitated by addition of about 900 mL ethanol. A cotton-like white solid was recovered by filtration and dried under vacuum overnight.

The HA-SODm solid was reprecipitated in ethanol, dried, and dissolved in 5 mM Tris buffer, pH 7.1, to a concentration of 15 mg/mL. Inductively coupled argon plasma (ICAP) analysis (Galbraith Laboratories, Knoxville, Tenn.) for elemental Mn showed a 2.7% binding of SODm 470 to HA.

The activity of the SODm in HA-SODm was measured by preparing 160 μL of 2 μM, 4 μM, 6 μM, 8 μM, or 10 μM HA-SODm in 60 mM HEPES buffer (Type 1 water, double-distilled, first from alkaline KMnO₄, then from EDTA), then rapidly mixing the solution with a potassium superoxide solution in dry HPLC grade DMSO (2 mM, 8 μL) using a computer controlled stopped-flow spectrophotometer. The absorbance decay of superoxide anion in the mixture was monitored at 250 nm, from 2 ms to ˜1 s after mixing. From a semilog plot of 1 nA vs. time, a first order k_{obs} was obtained for HA-SODm at each concentration. Catalytic activity of HA-SODm was demonstrated by the plot of k_{obs} vs. [HA-SODm] being linear throughout the concentration range. The slope of the line was equal to k_{cat}.

To measure kinematic viscosity of HA solutions, a Brookfield Engineering Laboratories model DV II+ viscometer with a CP52 spindle was used at 37° C. and 1 revolution/s. Kinematic viscosity measurements were performed at low shear, where molecular conformation and chain entanglements are present and contribute significantly to the measured viscosity. Viscosity measurement at low shear provides a more sensitive measure of changes in these factors and chain scission than does measurement at higher shear.

Sodium hyaluronate concentrations were determined by the modified carbazole method (Bitter et al., Anal. Biochem. 4:330 (1962)).

Size exclusion chromatography (SEC) was performed on a Waters Alliance Chromatographic system equipped with a 2487 UV detector at 280 nm and a Waters 410 refractive index detector. A TSK GMPW×1 column (7.8×300 mm) was used with a 150 mM NaNO₃ mobile phase at a flow rate of 0.8 mL/min. About 300 μL sample at about 0.1 mg NaHA/mL in the mobile phase was injected. Pullulan narrow molecular weight standards dissolved in water at 0.5 mg/mL were injected to create a calibration curve. Peak molecular weights of samples were determined relative to the pullulan standards using a first order equation for the calibration curve.

Results

The catalytic activities of both SODm 470 and SODm 470 bound in the HA-SODm complex were measured as described above. The catalytic rate constant k_{cat} of SODm 470 was measured to be 1.2×10⁷ M⁻¹ s⁻¹, and that for SODm 470 in the HA-SODm complex was measured to be 0.94×10⁷ M⁻¹ S⁻¹.

The kinematic viscosity of both HA and HA-SODm 470 were measured as described above. Sufficient NaCl was added to obtain an osmolality of 270-310 mOsm/kg. Results are as follows (Table I):

| Test | HA control | HA-SODm 470 |
|---|---|---|
| [NaHA] (mg/mL) | 14.4 | 20.2 |
| Kinematic viscosity (cps) | 16,100 | 14,940 |
| Intrinsic viscosity (mL/g) | 1840 | 1310 |
| Molecular weight from | 1.09 × 10⁶ | 7.00 × 10⁵ |
| intrinsic viscosity (Da) | | |
| pH | 7.0 | 7.1 |
| Osmolality (mOsm/kg) | 275 | 290 |
| Bioburden (colony forming | 0 | 0 |
| units/device) | | |

Free radical degradation of HA was investigated using the xanthine oxidase system to produce free radicals in the presence of HA. Experiments were performed directly in the viscometer to allow the real-time measurement of kinematic viscosity. Prior to measuring viscosity on the control HA solutions, 0.5 mL control HA solution was added to the viscometer cup, followed by 40 μL xanthine solution (20 mM), 10 μL EDTA solution (50 mM in Tris buffer), 10 μL xanthine oxidase solution (21.4 mg/mL in Tris buffer) or Tris buffer (control), and 10 μL SODm (2 mM in Tris buffer) or Tris buffer (control). All samples received the same volume dilution, and therefore differences in viscosity could be attributed to changes in the hyaluronate concentration. After addition of all solutions, the viscometer cup was placed on the viscometer and viscosity measurements taken every minute for 20 min. Measuring viscosity of HA-SODm solutions followed the same protocol, except that the EDTA solution was 100 mM, and 20 μL xanthine oxidase was used.

A control HA solution with no superoxide radical challenge showed no change in viscosity over the course of the experiment. A control HA solution with superoxide radical challenge showed rapid viscosity loss, viz., a 43% decrease in viscosity in 20 min. A solution of HA and SODm with superoxide radical challenge showed a protective effect of SODm on HA, showing a very slight loss of viscosity over 20 min. For HA-SODm solutions, only small changes in viscosity (less than about 10%) were observed over 20 min.

After viscosity experiments were performed, size exclusion chromatography (SEC) was performed on the HA and HA-SODm samples. The samples were removed from the viscometer, placed in microfuge containers, and frozen in a laboratory freezer. Prior to SEC, the samples were removed, thawed, and diluted in 50 mM bathocuproine (2,9-dimethyl-4,7-diphenyl-1,10-phenanthroline) to inhibit further enzymatic production of free radicals.

The SEC results were consistent with the kinematic viscosities reported above, as shown in Table II:

| | Relative peak MW |
|---|---|
| Sample | (pullulan standards) (MDa) |
| control HA | 8.7 |
| control HA with xanthine oxidase | 3.5 |
| challenge | |
| control HA with 2x xanthine oxidase | 3.5 |
| challenge | |
| control HA, xanthine oxidase challenge, | 7.5 |
| SODm 470 | |
| HA-SODm 470 with 2x xanthine oxidase | 4.2 |
| challenge (#1) | |
| HA-SODm 470 with 2x xanthine oxidase | 4.3 |
| challenge (#2) | |

The HA-SODm samples showed resistance to xanthine oxidase relative to control HA. It is believed the relative peak MW reduction of the HA-SODm samples relative to the HA/SODm 470 sample may have been due to changes in the conformation and hydrogen bonding of native HA, as hydrogen bonding in HA is believed to involve carboxylate groups, at least some of which are lost to functionalization with SODm 470. As a result, increased flexibility and decreased size and viscosity of HA are likely.

### EXAMPLE 2

The effects of HA and HA-SODm in New Zealand White rabbits with large surgical defects in trochlear articular cartilage were studied. This model exhibits rapid formation of osteophytes, one symptom of osteoarthritis.

Materials and Methods

HA (derived from rooster comb; Hyaluron Inc., Wayland, Mass.) and HA-SODm were prepared as described under Example 1. Sterile saline solutions were prepared as a negative control.

Five skeletally mature (6-9 months, 1.9-2.5 kg) New Zealand White rabbits of both sexes (Charles Rivers) received bilateral 6×3×2 mm defects drilled in the center of the trochlear groove using standard sterile operative procedures with halothane anesthesia and ketamine and xylazine premedication. The patella was displaced through a medial parapatellar approach to create the defects. Following surgery, the joint and overlying skin were closed with non-absorbable suture and the animals returned to standard housing without restriction of movement and with buprenorphin analgesia. Two days after surgery, each animal received a single 0.3 mL intraarticular injection of 1% (w/v) solution of HA in Tris buffer, pH 7.5, in one knee. The opposite knee received a single injection of a similar concentration and volume of HA containing 4 mg of covalently bound SODm (HA-SODm) in the same buffer. The injection sites were sterilized with soap and water and betadine prior to the infrapatellar injections using a 27-gauge needle attached to a tuberculin syringe. Animals received 1M injections of ketamine and xylazine for analgesia and sedation prior to intraarticular injections. Animals were given no post-injection analgesics to prevent obscuration of antiinflammatory and analgesic effects of HA and SODm.

A second group of 5 animals underwent identical procedures, but received three injections of HA-SODm in one knee, and three 0.3 mL injections of sterile phosphate buffered saline (PBS) in the opposite knee, with one injection in each series performed at day 2, 7, and 14. The knees injected with saline alone served as negative controls for single and multiple injections of test substances.

Both groups of animals were euthanized by phenobarbitol overdose 28 days after initial surgery. The knees were dissected and fixed in 10% neutral buffered formalin for 24 hr. Distal femurs were cut transversely into distal, central, and proximal slabs with a diamond wire saw. The central 6 mm slab included the entire trochlear defect, and the other two slabs included equal amounts of tissue proximal or distal to the defect. The slabs were decalcified and further fixed in a formic acid/citric acid decalcifying solution for 24-48 hr prior to paraffin embedding and sectioning at 5 μm. Sections were stained with hematoxylin, eosin, and Azure B at pH 4.5 with an alcoholic eosin counterstain. Cellularity, staining, and integrity of articular cartilage within and adjacent to the defects were evaluated qualitatively.

In addition, the overall trochlear composition and geometry was measured by histomorphometry. Using the Image 1 image analysis system, the following parameters were measured: (1) size and shape of trochlea and trochlear groove; (2) size of trochlear osteophytes in cross-sections through the middle of each trochlear slab; (3) percentage of cartilage, bone, and fibrous tissue in cross-sections through the middle of each trochlear slab; and (4) trabecular bone content of distal trochlea in cross-section through the middle of the defect.

Data were analyzed by 1-way ANOVA with post-hoc comparisons by the Tukey and Scheffe procedures, by Kruskal-Wallis 1-way ANOVA by ranks with post-hoc comparisons by Kruskal-Wallis Z-test, and by single sample paired T-tests using programs of the Number Cruncher and Power Analysis Statistical Systems, Kayesville, Utah.

Results

No adverse event (such as death, infection, weight loss, joint effusion, or fever) occurred in any of the animals, and thus no animals were removed from the study for any reason. Macroscopic evaluation of the articular cartilage surface and the synovium revealed no apparent differences between HA-SODm treated knees and control knees.

As expected, the large defects failed to heal within the 28 day test period. The defects were filled with variable amounts of cartilage, fibrous tissue, and bone, and were often poorly integrated with the surrounding cartilage and bone and had deep clefts in their surfaces. Defects were slightly smaller and occupied a smaller proportion of the trochlea in joints injected with HA-SODm than in joints injected with HA alone, but the same cartilage content was seen with both treatments. Trochlear size and the percentage composition of bone and cartilage were the same in the two treatment groups, as shown in Table III. Also, articular cartilage adjacent to the defects was relatively normal in both treatment groups.

TABLE III

| | | | Paired Sample | Paired Sample |
|---|---|---|---|---|
| Parameter | HA | HA-SODm | T-Test - T | T-Test - P |
| Trochlear | 139 ± 7 | 136 ± 9 | 0.44 | 0.69 |
| size (mm²) | | | | |
| Bone (% | 38 ± 4 | 33 ± 8 | 1.06 | 0.36 |
| trochlear | | | | |
| area) | | | | |
| Cartilage (% | 5 ± 0.4 | 6 ± 0.8 | 0.69 | 0.53 |
| trochlear | | | | |
| area) | | | | |
| Defect area | 17 ± 2 | 10 ± 1 | 3.23 | 0.02 |
| (mm²) | | | | |
| Defect (% | 12 ± 2 | 4 ± 1 | 2.57 | 0.04 |
| trochlear | | | | |
| area) | | | | |
| Cartilage (% | 18 ± 5 | 16 ± 4 | 0.22 | 0.84 |
| defect area) | | | | |

Trochlear geometry was evaluated by measuring lines and angles related to the shape and size of the trochlear groove at the level of the articular cartilage surface and at the level of the subchondral bone surface. Although all the knees with trochlear defects had alterations relative to normal trochlea, there were no significant differences in mean or median values of any of the lines or angles between knees treated with single injections of HA and knees treated with single injections of HA-SODm.

A major finding was a significant variation in osteophyte size among the different treatment groups (Table IV). Analysis by one-way ANOVA showed that the mean size of osteophytes in joints receiving a single injection of HA was significantly larger than those in animals receiving single or triple injections of HA-SODm. Otherwise, ANOVA showed no significant differences among joints treated with single or triple injections of saline or HA-SODm. However, the same data compared by two-sample T-tests showed significant differences between joints treated with a single injection of HA and all of the other treatments, including saline injections. Also, this analysis showed larger osteophytes in saline-injected joints than in those receiving a single injection of HA-SODm.

TABLE IV

| | Osteophyte | | | | | |
|---|---|---|---|---|---|---|
| | Area | 1-Way ANOVA | | | | |
| Treat- | (mm²) | F | P | Power | T-Tests | |
| ment | Mean ± ISD | (6.91) | (0.004) | (0.92) | T | P |

| | | | | |
|---|---|---|---|---|
| r-HA | 11.0 ± 6.3 | 1 > 2, 3, 4 | 1 > 2, | 0.01-0.03 |
| 1x | | | 3, 4 | |
| r-HA- | 1.1 ± 0.4 | 1 > 2 = 3, 4 | 3 = | 0.01-0.03 |
| SODm | | | 2 < 1, 4 | |
| 1x | | | | |
| r-HA- | 2.3 ± 0.7 | 2, 4 = 3 < 1 | 1 > 3 = | 0.01 |
| SODm | | | 2, 4 | |
| 3x | | | | |
| saline | 4.4 ± 1.2 | 4 = 1, 2, 3 | 1 > 4 > | 0.01-0.03 |
| 3x | | | 2 | |

Discussion

One finding of this study was data suggesting that HA may increase the mean size of osteophytes and that HA-SODm reverses this process. Also, HA-SODm decreases osteophyte formation relative to animals injected with saline. Increased osteophyte scores in comparison to saline injected controls have been reported in the knees of meniscectomized sheep injected with HA. The finding in this study that covalent binding of SODm to HA significantly reduces the mean size of osteophytes in rabbits injected with HA or saline suggests that SODm has beneficial effects in preventing both baseline and accelerated osteophyte formation. It is possible that osteophyte formation is accelerated by HA degradation products, and that SODm may retard that process. In any case, the data indicate a favorable response to HA-SODm injections in the rabbit model.

To summarize, HA-SODm, either with single or triple injection, significantly reduced osteophyte size compared to saline or HA controls. Thus, a stabilized HA product can reduce a symptom of osteoarthritis.

Second, there were no significant differences between single or triple HA-SODm injections on osteophyte size. Thus, a single HA-SODm injection demonstrated efficacy comparable to a triple injection.

Third, HA alone significantly increased osteophyte size compared to the saline control. Thus, whereas degraded HA increased osteophyte size, HA-SODm (HA protected from degradation) decreased osteophyte size.

### EXAMPLE 3

A biocompatibility study was performed, involving direct injection of SODm 403 into canine knees.

Male and female dogs (purebred beagles, Covance Research Products Inc., Madison, Wis., age about 8 months to 12 months, weight about 9 kg to 12 kg) were assigned to three groups, as described in Table V. The right stifle joint of each animal was dosed intraarticularly with vehicle (sterile saline), and the left stifle joint of each animal was dosed intraarticularly with a solution of SODm 403 in sterile saline at a particular dose level.

TABLE V

| | | | target dose | | |
|---|---|---|---|---|---|
| | # male | # female | level | dose volume | samples |
| Group # | animals | animals | (mg/animal) | (mL/animal) | collected |
| 1 | 1 | 1 | 4 | 1 | blood/tissue |
| 2 | 1 | 1 | 13 | 1 | blood/tissue |
| 3 | 1 | 1 | 40 | 1 | blood/tissue |

Dose administration involved the use of sterile filtered solutions. Dose administrators wore sterile surgical gloves during dosing. The appropriate solution (vehicle or test solution) was drawn into a syringe with an attached 1-in, 20 gauge thin-walled needle. The area was prepared aseptically, and the dogs were anesthetized with isoflurane and maintained throughout the dosing procedure. The midpoint of the patellar tendon, between the patella and the tibial crest, was palpated, and the needle introduced into the joint, entering the skin immediately lateral to the midpoint of the patellar tendon. The needle was directed to the intercondylar space and the dose was slowly injected over a period of approximately 30-40 s. The needle was withdrawn from the joint, and the area immediately covered with a gauze sponge soaked in povidone-iodine solution and held in place for approximately 1 min.

Mortality, moribundity, health and appearance observations, and observations for joint stiffness were done 1×-2×daily. The Group 3 male appeared stiff at both stifle joints for 48 hr postdose. The Group 3 female did not use the left hind leg at 24 hr postdose and had swelling at the joint through 48 hr postdose. Blood (approximately 3 mL) was collected via a jugular vein into tubes containing sodium heparin at 0.5 hr and 2 hr following the dose to the left stifle joint. At 72 hr following the dose to the left stifle joint, each animal was euthanized by exsanguination, and the following tissues were collected from the stifle joint of each hind limb and preserved in 10% neutral-buffered formalin: articular cartilage, meniscus, patella, patella ligaments, popliteal lymph node, and proximal tibia. Tissues were embedded in paraffin, sectioned, stained with hematoxylin and eosin, and examined microscopically.

The only microscopic finding that may be related to test material administration was a minimal hypertrophy of synovial cells in the section of the left meniscus of the Group 2 and Group 3 male and female animals.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1-6. (Canceled)

7. A method of reducing the mean size of osteophytes in a joint of a mammal, comprising:
(i) providing a composition, comprising a superoxide dismutase mimic (SODm) associated with hyaluronic acid (HA), wherein the composition is a liquid at about 37° C.; and,
(ii) injecting the composition into or close to the joint.

8. The method of claim 7, wherein the mammal is a human.

9. The method of claim 7, wherein the joint is a knee.

10. The method of claim 9, wherein the composition is injected into the knee.

11. The method of claim 7, wherein the SODm is associated with the HA by a chemical bond.

12. The method of claim 11, wherein the chemical bond is selected from the group consisting of a covalent bond and an ionic bond.

13. The method of claim 12, wherein the chemical bond is a covalent bond and the covalent bond is selected from the group consisting of an amide bond, an ester bond, and an ether bond.

14. The method of claim 7, wherein the SODm has structure I: 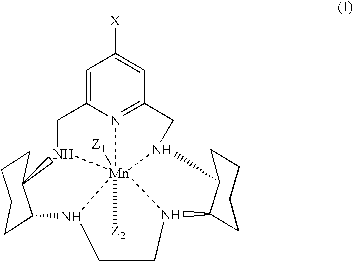
wherein X is selected from the group consisting moieties comprising —H, moieties comprising —NH₂, moieties comprising —COOH, and moieties comprising —CHOH; and Z₁ and Z₂ are independently -halide.

15. The method of claim 14, wherein X is —S—(CH₂)₂—NH₂, and Z₁ and Z₂ are each —Cl.

16. The method of claim 7, wherein the dose of SODm is from about 0.01 mg SODm/kg body weight to about 10 mg SODm/kg body weight.
